(19) United States
(12) Patent Application Publication
(10) Pub. No.: US 2026/0155221 A1
(43) Pub. Date: Jun. 4, 2026

(54) METHOD AND APPARATUS FOR DATA INPUT FOR DIGITAL HEALTH COMMUNICATION SYSTEM AND PREPARATION OF A REPORT OF PATIENT HEALTH USING ARTIFICIAL INTELLIGENCE AND MACHING LEARNING

(71) Applicant: David Scott DuPlay, Flemington, NJ (US)

(72) Inventor: David Scott DuPlay, Flemington, NJ (US)

(21) Appl. No.: 19/326,682

(22) Filed: Sep. 11, 2025

Related U.S. Application Data
(63) Continuation-in-part of application No. 18/968,034, filed on Dec. 4, 2024.

(51) Int. Cl. G16H 15/00; G06F 16/35; G16H 50/30

(52) U.S. Cl. CPC G16H 15/00; G06F 16/35; G16H 50/30

(57) ABSTRACT

Embodiments provide for data input methods and systems for computer record storage and retrieval. In embodiments, an artificial intelligence system or a computer-based nodal analysis system allows for scanning or document acceptance, sorting, deletion of duplicate materials, storage and retrieval of data for use by individuals, such as health care professionals. Access to the methods and systems may be monitored or allowed according to different criteria such as the satisfaction of a payment criteria.

### CROSS-REFERENCE TO RELATED APPLICATIONS

The current application claims priority to, and is a continuation-in-part application of, application Ser. No. 18/968,034 filed Dec. 4, 2024, the entirety of which is incorporated by reference.

### FIELD OF INVENTION

Aspects of the disclosure relate to methods and devices for health care tracking, prediction, communication, and health care plan development wherein the communication may involve one person who may be elderly or under the care of a physician other healthcare professional or allied health professional. Aspects further relate to an artificial intelligence system that may use devices which permit constant supervision and monitoring of a person by other people or entities. Aspects of the system monitor data obtained from a data source and provide insights to a patient's wellbeing as well as future care plans for the patient. Further aspects of the disclosure provide for assessing a medical condition of an individual as compared to a general community as selected by a series of parameters by a user and/or an artificial intelligence system.

Aspects of the disclosure also relate to methods and devices for data input, storage, normalization, and retrieval from different mediums. Such mediums can include hand-written notes, digital files, pictures, photographs, graphs, charts, and reports as non-limiting embodiments. Data obtained through input may be processed through an artificial intelligence or machine learning network to eliminate redundancy, provide analysis, and produce an aging-in-place score for a person to establish a snapshot of a patient's health and potential future health care needs based upon health care date.

### BACKGROUND INFORMATION

People are increasingly relying upon technology to stay connected with loved ones, their peers, healthcare providers, etc. With systems such as email, text messages and video conferencing becoming mainstream, formerly conventional methods of correspondence are being used less frequently. This is, in part, because technology allows people to connect more quickly and share more information than conventional methods. For example, using technology, pictures, audio files, and even video may be transferred and received with people at the touch of a button. Even medical or service providers and benefits may be managed primarily online.

Health care patients often present with complex health care issues that require multifaceted approaches to manage effectively. These complexities arise from a multitude of conditions that can coexist, interact, and evolve over time, necessitating continuous monitoring and tailored or personalized interventions. Patients may display different characteristics, such as varying symptoms, reactions to treatments, and progressions of diseases, that can be collected as data. This data can provide invaluable insights into their health status and inform critical health care decisions. However, the sheer volume of data available poses significant challenges to effectively harness it for meaningful analysis.

The effectiveness of conventional analysis techniques is often constrained by the limitations of in-person doctor assessments. While face-to-face consultations remain a cornerstone of health care, they are not always feasible for every patient. Elderly patients, in particular, may face significant barriers in accessing medical care due to mobility issues, geographic limitations, or the need for continuous supervision. This can lead to gaps in care and missed opportunities for early intervention and optimal health management. As a result, relying solely on traditional methods can hinder comprehensive health care delivery and outcomes.

There is a pressing need to develop systems capable of evaluating data for patients from multiple sources. Integrating data from various databases allows for a more holistic view of a patient's health, encompassing a wider range of factors that influence or determine their well-being. Such systems must be able to collate and analyze data efficiently, providing health care professionals with actionable insights that drive better patient outcomes. This approach ensures that care plans are both responsive and adaptive to the evolving needs of patients.

Furthermore, there is a need to utilize different types of databases to create comprehensive care plans and accurately predict future health care needs. Databases such as insurance data, healthcare records, patient testing results, clinical lab results, diagnosis code data, treatment code data, physical therapy data, occupational therapy data, actuarial tables, and pharmacy records offer diverse and complementary information. By leveraging these data sources, health care systems can develop more precise and personalized care plans. This not only enhances the quality of care but also enables proactive management of potential health issues, thereby improving long-term health outcomes for patients.

The integration of advanced data analysis techniques and diverse data sources is essential in addressing the complex health care issues faced by patients today. By overcoming the limitations of conventional methods and embracing innovative approaches, health care providers can offer more effective, personalized, and anticipatory care.

The current population of the United States has approximately 30 million individuals over the age of 75. This number is expected to increase to over 100 million individuals in the next 20 years. Traditional families with three to four children are being replaced with lesser numbers of children. As a result, the children that are born to families in the future will often have extended care responsibilities for parents.

To handle the increase in numbers of aging individuals, society will need to increase the number of assisted living and memory care communities. While increases are expected, the number of older adults is still expected to outstrip the number of possible vacancies in assisted living communities. There is a need; therefore, to help treat, care and monitor individuals that are both in assisted living communities as well as at home or other care facilities.

In today's healthcare landscape, multiple database sources contain critical health information about patients. These sources include insurance records, healthcare databases, patient testing results, actuarial tables, and pharmacy records. Each database offers a unique set of data, contributing crucial insights into a patient's overall health. The information within these databases often remains siloed, leading to fragmented health data that is difficult to collate and analyze comprehensively. This fragmentation presents significant challenges for healthcare providers who strive to deliver personalized and effective care based on a holistic view of the patient's health status.

Despite the wealth of data available, there is a notable absence of a single, integrated system that can seamlessly analyze information from these diverse sources. Currently, healthcare professionals and institutions rely on various platforms and tools to interpret patient data, which can be time-consuming and inefficient and lack leading or real-time indicators of health. The lack of a unified system hampers the ability to provide an accurate and up-to-date status of an individual's health. More critically, it limits the capacity to predict future healthcare needs, which is essential for proactive and preventative care. Without a consolidated approach, healthcare providers may miss opportunities for early intervention and optimized health management.

Conventionally, doctors do not integrate and process data from multiple healthcare databases but rather conduct a physical exam and immediately order follow-up tests or potential remedies. Conventional healthcare approaches do not provide comprehensive insights into a patient's current health status and anticipate future health needs. No single healthcare services platform provides for an accurate predictive analysis of patients in order to tailor care plans that are both responsive and adaptive to the evolving health conditions and aging process of patients.

Health care outcomes for patients are often determined not only by their health conditions but also by the economic resources available to them. Patients with limited financial means frequently encounter barriers to accessing necessary medical services and treatments. This economic disparity leads to significant differences in health outcomes, with wealthier individuals typically receiving more comprehensive and timely care. Economic constraints can limit a patient's ability to afford medications, diagnostic tests, and follow-up appointments, which are crucial for effective health management and disease prevention.

Conventional health care procedures often focus on addressing current symptoms, with little attention or ongoing management given to long-term planning for future health care needs. This reactive approach can be detrimental, particularly for individuals who lack the financial means to access preventative services and early interventions. Patients without the resources to afford personal doctors or extensive health care plans often miss out on opportunities to identify and manage potential health issues before they become severe. This lack of foresight in health care planning disproportionately affects economically disadvantaged individuals, leading to poorer health outcomes and higher medical costs over time.

There is an urgent need to develop and implement health care plans that cater to individuals who are less economically well-off. These plans must be both accessible and accurate, ensuring that all patients receive the necessary care to maintain and improve their health. By incorporating predictive analytics and advanced data integration techniques, health care providers can create personalized care plans that account for a patient's current conditions and anticipate future health care needs. This proactive approach not only enhances the quality of care for economically disadvantaged patients but also supports future family planning by providing reliable health care strategies that can adapt to changing circumstances.

As the number of patients increases worldwide, the caregivers for these patients will be forced to care for larger numbers of patients or, non-skilled (home) caregivers may be used to satisfy the needs of patients. In the case of both skilled and non-skilled caregivers, stresses on such individuals tending to the needs of others increases. Symptoms such as higher blood pressure, lack of sleep and other signs of stress are likely to increase. Conventionally, no system records the well-being of the caregiver as well as the patient. Often, the caregiver is taken as a hired professional or a spouse/family member that will have to deal with the struggles of care, ignoring the family wide implications of the provided care. Social isolation may occur for both the patient and the caregiver as long hours involved with treatment may be experienced.

Differing types of systems are used in creating data for those being cared for at home or in a care facility. These systems can include, but are not limited to, handwritten notes, charts, computer emails, prescription materials, photos from mobile phones, photos from digital cameras, voice through national language processing software, voice through national language processing devices, remote patient monitoring devices, and medical tests. Each one of these systems may use a different format and as such, accumulating such data is cumbersome. While accumulation of such data is performed in a rudimentary basis, the propensity to miss data that has been gathered is more prevalent when a patient has several sources of data. In instances where a patient is in an extended care facility or has a long medical history, the number of records that exists may exponentially increase, making accurate diagnosis difficult and in many cases impossible. Currently, there are no systems that can accurately accumulate desired data into a single platform for review and analysis for care plan recommendations

To further the problems encountered for care with individuals, medical professionals and extended care teams from home care and home health agencies are often changed throughout the lifetime of a care recipient. Medical professionals and extended care teams may change geographic locations, patients may move geographic locations, medical professionals and extended care professionals may retire or switch positions. Except in very rare and limited cases, a patient may have dozens of care professionals during their lifetime. There is no centralized system that follows a patient with the data needed for diagnosis and dynamic care plan management. To counter this, conventional practice for medical professionals is to do a complete and exhaustive series of tests to create a baseline health care prognosis for the patient. This causes unneeded tests for the patient as well as economic costs. With the rising cost of healthcare, such economic costs can be economically prohibitive for a patient.

Conventional systems do not provide a system that provides a summary or a benchmark of a patient's data compared to a norm. Because the different types of data are not combined into a single system, an accurate assessment of the patient is not provided to medical care providers. To supplement the lack of complete data, medical professionals utilize past experiences to help in patient treatment. When a medical condition is experienced that the practitioner is not familiar with is encountered, the chances of misdiagnosis are greatly increased.

As the care providers (doctors, home care workers, home health professionals, and staff) may change, early identification of patient health decline may go unnoticed as new medical professionals and extended care teams without individual patient experience are incorporated.

There is a need to be able to accurately prepare a health care action plan for patients that is reliable.

There is a further need to be able to quickly assess a patient's data and to be able to easily add proprietary databases into an analysis system that will summarize and predict patient health, wellness or outcomes.

There is a further need to be able to process patient data in a cost-effective way to minimize medical professional resources while providing an accurate assessment of patient condition.

There is a further need to be able to track a caregiver's health in addition to the patient to identify and/or prevent medical complications from occurring in others, aside from the patient, as this tracking has a direct impact on the care that the caregiver can provide to the patient.

As a patient ages, there are often new needs that are encountered for treatment. Prescreening of patients is a requirement for many types of tests. There is a further need to allow for prescreening of patients for medical tests to simplify the administration of medical tests and costs.

There is a need to provide a data accumulation system that will take various forms of medical records, health information, and wellness information and store the data contained therein.

There is a further need to provide a data accumulation system that will provide for long-term safe and secure storage of such data.

There is a further need to provide for a system to convert the various forms of data into a structured format or database.

There is a further need to provide for making the structured format or database searchable by medical professionals.

There is a further need to provide data driven decisions for health and wellness care, and medical care that can anticipate or diagnose symptoms a patient may have that is outside of the experience of the medical professional.

There is a further need to provide for a system that will identify underperformance in various medical conditions compared to a norm and determine if the underperformance is related to a lack of care aide performance.

As a corollary to the above, there is a further need to provide for a system that will identify overperformance in various medical conditions, and normal aging compared to a norm, and determine when the overperformance is related to superior care aide performance.

There is a further need to provide a system that provides detection of patient health decline that is more independent from medical professionals, thus allowing newer medical professionals to ascertain changes in patient status in a quick and timely manner.

### SUMMARY OF THE INVENTION

So that the manner in which the above recited features of the present disclosure can be understood in detail, a more particular description of the disclosure, briefly summarized below, may be had by reference to embodiments, some of which are illustrated in the drawings. It is to be noted that the drawings illustrate only typical embodiments of this disclosure and are; therefore, not to be considered limiting of its scope, for the disclosure may admit to other equally effective embodiments without specific recitation. Accordingly, the following summary provides just a few aspects of the description and should not be used to limit the described embodiments to a single concept.

In one example embodiment, a method to produce a health care plan for an individual is disclosed. The method may comprise inputting data related to an individual, the data related to at least one health care statistic for the individual. The method may further comprise performing a quality check of the input data related to the individual. The method may further comprise looping back to the inputting data related to the individual when the quality check indicates that a threshold amount of data of sufficient quality has not been satisfied. The method may further comprise accessing an independent artificial intelligence program. The method may further comprise performing an authentication check of the accessing of the artificial intelligence program to verify that the accessing possesses sufficient authentication for artificial intelligence access. The method may further comprise ending the method when the authentication check to the artificial intelligence fails. The method may further comprise accessing at least one database that contains data related to healthcare patients other than the individual. The method may further comprise creating a list of data to be searched with the at least one database based upon input data from the individual. The method may further comprise searching the at least one database based upon the list of data to be searched to find similar individuals having data within the at least one database and retaining the matching data. The method may also comprise removing individual personal identifying information from the matching data. The method may further comprise using the artificial intelligence program to sort and analyze the matching data to identify a current healthcare status for the individual. The method may further comprise using the current healthcare status and risk for the individual and the matching data, identifying likely future healthcare outcomes for the individual. The method may further comprise creating the healthcare plan for the individual based on the likely future healthcare outcomes for the individual. The method may further comprise at least one of displaying and saving the health care plan for the individual.

In another example embodiment, a method to determine a health care status of at least one individual is disclosed. The method may comprise inputting data related to the at least one individual, the data related to at least one health care statistic for the individual. The method may further comprise performing a quality check of the input data related to the individual. The method may further comprise looping back to the inputting data related to the individual when the quality check indicates that a threshold amount of data of sufficient quality has not been satisfied. The method may further comprise accessing an independent artificial intelligence program. The method may further comprise performing an authentication check of the accessing of the artificial intelligence program to verify that the accessing possesses sufficient authentication for artificial intelligence access. The method may further comprise ending the method when the authentication check to the artificial intelligence fails. The method may further comprise accessing at least one database that contains data related to healthcare patients other than the individual. The method may further comprise creating a list of data to be searched with the at least one database based upon input data from the individual. The method may further comprise searching the at least one database based upon the list of data to be searched to find similar individuals having data withing the at least one database and retaining the matching data. The method may further comprise removing individual personal identifying information from the matching data. The method may further comprise using the artificial intelligence program to sort and analyze the matching data to identify a current healthcare status for the individual.

In another example embodiment, a method to determine a health care status of at least one individual is disclosed. The method may comprise accessing an independent artificial intelligence program. The method may also comprise accessing at least one database that contains data related to healthcare patients other than the individual. The method may also comprise selecting health care data for the at least one individual. The method may also comprise creating a list of individuals with similar health care data to the at least one individual. The method may also comprise using the artificial intelligence program to sort and analyze the matching data to identify a current healthcare status for the individual to achieve a result. The method may also comprise at least one of displaying and printing the result.

In another example embodiment, a method of processing medical data is disclosed. The method may comprise obtaining a first set of medical data related to a first medium. The method may further comprise obtaining a second set of medical data related to a second medium. The method may further comprise inputting the first set of medical data into an artificial intelligence, machine learning, and neural network program. The method may further comprise evaluating the first set of medical data with the artificial intelligence, machine learning, and neural network program to produce first results. The method may further comprise inputting the second set of medical data into the artificial intelligence, machine learning, and neural network program. The method may further comprise evaluating the second set of medical data into the artificial intelligence, machine learning, and neural network program to produce second results. The method may further comprise sorting the first results and the second results into database categories. The method may further comprise storing the first results and the second results into a database. The method may further comprise extracting data from the database into a modeling database. The method may further comprise evaluating the modeling database with the artificial intelligence system to produce evaluated results. The method may further comprise producing at least one report with the evaluated results.

In another example embodiment, a method of processing health, wellness, and medical data is disclosed. The method may comprise inputting a first set of medical data into an artificial intelligence, machine learning, and neural network program. The method may further comprise evaluating the first set of medical data with the artificial intelligence, machine learning, and neural network program to produce first results. The method may further comprise sorting the first results into database categories. The method may further comprise extracting data from the sorted first results into a modeling database. The method may further comprise evaluating the modeling database with the artificial intelligence, machine learning, and neural network system to produce evaluated results. The method may further comprise producing an aging in place score for a patient.

In another example embodiment, an article of manufacture, containing a list of instructions that is readable by a computing apparatus is disclosed. The list of instructions, at least in part, comprising: a method of processing medical data, comprising obtaining a first set of medical data related to a first medium. The article of manufacture having the list of instructions further has a step of obtaining a second set of medical data related to a second medium. The article of manufacture having the list of instructions further has a step of inputting the first set of medical data into an artificial intelligence program. The article of manufacture having the list of instructions further has a step of evaluating the first set of medical data with the artificial intelligence program to produce first results. The article of manufacture having the list of instructions further has a step of inputting the second set of medical data into the artificial intelligence program. The article of manufacture having the list of instructions further has a step of evaluating the second set of medical data into the artificial intelligence program to produce second results. The article of manufacture having the list of instructions further has a step of sorting the first results and the second results into database categories. The article of manufacture having the list of instructions further has a step of storing the first results and the second results into a database. The article of manufacture having the list of instructions further has a step of extracting data from the database into a modeling database. The article of manufacture having the list of instructions further has a step of evaluating the modeling database with the artificial intelligence system to produce evaluated results. The article of manufacture having the list of instructions further has a step of producing at least one report with the evaluated results.

### BRIEF DESCRIPTION OF THE FIGURES

FIG. 1 illustrates a system implementation footprint for databases used by an artificial intelligence system in one example embodiment of the disclosure.

FIG. 2 illustrates a voice biomarker speech assessment by an artificial intelligence system and machine learning through biomarker data.

FIG. 3 is an illustration of a user ecosystem, app store and partner organization.

FIG. 4 is an illustration of the structured, semi-structured and non-structured database components being fed into the artificial intelligence system and machine learning platform.

FIG. 5 is an illustration identifying data sources an artificial intelligence and machine learning system, and stakeholder applications and production of charts, status and predictions for a patient and/or caregiver in one example embodiment of the disclosure.

FIG. 6 is a method of creating a dynamic care plan for a patient in one example embodiment of the disclosure.

FIG. 7 is a method to determine a health care status of at least one individual.

FIG. 8 is a method to determine a health care status of at least one individual.

FIG. 9 illustrates an ecosystem of different modules that interface with an artificial intelligence, machine learning, and neural network system, including a data insertion and conversion module, an agency performance management module, a care aid performance module, a smart care modeling module and a virtual care module.

FIG. 10 illustrates a data digitization, normalization and storage process in accordance with one example embodiment of the disclosure.

FIG. 11 illustrates one example embodiment method of processing medical data in accordance with the disclosure.

FIG. 12 illustrates another example embodiment method of processing medical data in accordance with the disclosure.

To facilitate understanding, identical reference numerals have been used, where possible, to designate identical elements that are common to the figures (“FIGS”). It is contemplated that elements disclosed in one embodiment may be beneficially utilized on other embodiments without specific recitation.

### DETAILED DESCRIPTION OF THE INVENTION

In the following, reference is made to embodiments of the disclosure. It should be understood; however, that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the following aspects, features, embodiments, and advantages are merely illustrative and are not considered elements or limitations of the claims except where explicitly recited in a claim. Likewise, reference to “the disclosure” shall not be construed as a generalization of inventive subject matter disclosed herein and should not be considered to be an element or limitation of the claims except where explicitly recited in a claim.

Although the terms first, second, third, etc., may be used herein to describe various elements, components, regions, layers and/or sections, these elements, components, regions, layers and/or sections should not be limited by these terms. These terms may be only used to distinguish one element, components, region, layer or section from another region, layer, or section. Terms such as “first”, “second”, and other numerical terms, when used herein, do not imply a sequence or order unless clearly indicated by the context. Thus, a first element, component, region, layer, or section discussed herein could be termed a second element, component, region, layer, or section without departing from the teachings of the example embodiments.

When an element or layer is referred to as being “on”, “engaged to”, “connected to”, or “coupled to” another element or layer, it may be directly on, engaged, connected, coupled to the other element or layer, or interleaving elements or layers may be present. In contrast, when an element is referred to as being “directly on”, “directly engaged to”, “directly connected to”, or “directly coupled to” another element or layer, there may be no interleaving elements or layers present. Other words used to describe the relationship between elements should be interpreted in a like fashion. As used herein, the term “and/or” includes any and all combinations of one or more of the associated listed terms.

Some embodiments will now be described with reference to the figures. Like elements in the various figures will be referenced with like numbers for consistency. In the following description, numerous details are set forth to provide an understanding of various embodiments and/or features. It will be understood; however, by those skilled in the art, that some embodiments may be practiced without many of these details, and that numerous variations or modifications from the described embodiments are possible. As used herein, the terms “above” and “below”, “up” and “down”, “upper” and “lower”, “upwardly” and “downwardly”, and other like terms indicating relative positions above or below a given point are used in this description to more clearly describe certain embodiments.

Aspects of the disclosure provide a system that uses artificial intelligence to prepare a dynamic care plan for an individual. The dynamic care plan may detail health care instructions/treatments and activities regarding health care needs of an individual. The dynamic care plan may be generated through processing of data from a single or multiple sources pertaining to the patient. Data may be input into the system through various means, such as a digital health device, a cell phone, a computing device, biometric sensors, insurance company databases, health record databases, voice scanning technologies, visual scanning technologies, patient testing data and other sources. Such described databases should not be considered limiting. Output of the analysis by the artificial intelligence system may be provided through a digital health device, cell phone, or other computing device. In embodiments, different individuals may be authorized to receive updated plans, such as a doctor, nurse, family member and/or patient. The artificial intelligence system may create the dynamic care plan through collection of data and health analytics within the digital health companion device as well as other resources. The other resources may include various external databases. Databases used may be added or subtracted from analysis by the artificial intelligence system. One non-limiting example may include analysis of healthcare and direct data input performed for a first individual. A second non-related patient may have a healthcare, insurance, and direct data input. The artificial intelligence system may be created to weigh different data sources so that appropriate data is analyzed. In some embodiments, a patient or a health care service may find that analyzing a greater number of databases results in more accurate and cost-effective care for the patient, therefore a subscription to a choice of databases may be created for the user. In other embodiments, a user may be an insurance company where data from a patient is input and various databases are used by the artificial intelligence system to create a dynamic care plan. This created dynamic care plan may be cross-referenced with actual care planned for the patient. If the two plans match, the insurance company may authorize treatment of the patient through the insurance company. If there is a discrepancy, further inquiry may be made as to the amount of the discrepancy and the ultimate costs of the planned activities.

The data may include social determinants of health analysis, as well as a population health analysis; Medicare and/or Medicaid care plan reporting, which may include a minimum data sheet. The data may further include the identification of patterns and trends at the individual level as well as the collective level, within the community and cross-sectional across populations, as well as a healthcare professional patient report review for insurance billing. In one embodiment, the dynamic care plan includes artificial intelligence (“AI”) enhanced data analytics and related care plan modifications. Aspects of the disclosure also provide for monitoring of a caregiver for patients. Aspects related to monitoring a caregiver may identify health conditions for caregivers who provide services to patients. Studies have shown that health caregivers, such as family members, are at a significantly increased higher risk of developing medical conditions based upon the stress and constant care requirements for some patients. Aspects of the disclosure provide for monitoring, through wearable technology and/or data input of the caregiver into an AI system.

Aspects of the disclosure includes providing a patient or a first person with a digital health companion and providing one or more caregivers or second and/or third persons with a companion app for use on their cellular phone, satellite phone, or computer. Other embodiments provide for data input through automatic means, such as wearable technology. Aspects of the disclosure provide for processing data from the above input means and providing a dynamic care plan for individuals, such as patients. Such dynamic care plans may include schedules for healthcare, expected costs, anticipated future events, longevity analysis and general health monitoring for both patients and caregivers. For data input through a digital health companion, potential customers include a full range from single community settings (home use) to multi-community settings. Aspects of the disclosure may be used in senior living communities, assisted living facilities, memory care facilities, continuing care retirement communities (“CCRCs”) and in-home care agencies and individuals. The digital health companion provides an efficient and effective way to stay connected to clients and their families 24 hours per day and 7 days per week. The digital health companion has been designed from the ground up for non-technical aging adults. Aspects of the disclosure also provide for input of data through cell phone applications, laptop computers, dedicated kiosks and other computing devices.

In one embodiment, the data is protected to allow for restricted access. Such restricted access is important as local and other governmental regulations may require strict control of access to patient data. Processing and communication may be protected, such as through a password, biomarker or encryption technology. In aspects using a dedicated device, the device may have several components. In some embodiments a sensor may be used to authenticate a user. This authentication may be through face recognition, fingerprint scan, hand scan, voice identification and/or password. Other security protection schemes are also possible and the above listed alternatives are but one possible embodiment. Input or communication devices may be provided with at least one audio speaker or video display for interaction with the patient. When a patient may require other input devices, such devices may be provided. In the case of visually impaired individuals, Braille keyboards and sound devices may be used. When a video display is provided, appropriate video and/or snap-shot capabilities for identification are possible. In embodiments, the artificial intelligence system is based on a server. The program may be monitored at a central location by engineers/operators to monitor the health of the computer system during operations. When equipped with a server, the artificial intelligence may access different databases for performance of calculations. In embodiments, the programming may allow for executing a protocol of instructions and controlling the transmission of signal through the server as well as back and forth to patients and caregivers. Other entities may also be given access to the AI system, such as insurance companies, healthcare providers and designated family members as non-limiting embodiments.

In certain embodiments, the protocol of instructions for the AI system may comprise one or more of the following: sending health-related reminders to a patient, sending health-care related reminders to a family member, verifying appointments for a patient, sending health and wellness education information to the user; sending cognitive tools to a device, such as a cell phone, kiosk, or dedicated device for the patient to hear, see or interact with. Other actions may include sending questions to the patient regarding health and wellness for the first person to hear, see or respond to.

In one embodiment, the protocol of instructions further comprises generating weekly reports of the activity on the device. In another embodiment, the protocol of instructions further comprises monitoring and analyzing the activities of the user on a device, modifying the instructions and generating a modified care plan.

In one embodiment, aspects of the disclosure provide an AI system included into a dedicated device directly as opposed to being located on a server. In still further embodiments, the AI system may be installed on a home computer, mobile computer or cellular telephone. These embodiments may use artificial intelligence (“AI”) and machine learning (“ML”) for monitoring and analyzing data. The data may be input directly on to a device or the AI system may access remote databases.

Aspects of the disclosure includes incorporating the AI system on a server, on a computing device and/or both. Aspects may be provided where a smaller scale AI system is provided on a hand-held device that interacts with a more complex AI system configured on a server. Each of the AI systems may be established to interact with a designated set of databases. This set of databases may be selected by a computer operator or custodian. For example, in the case of a mobile cell phone AI system, this system may be configured to only interact with a server-based AI system. The server-based AI system may contain a multitude of access capabilities to outside databases.

The AI system is designed to provide secure portal access to insights using AI and ML for aggregating global resident, patient and caregiver insights across all chronic, terminal and rare diseases to advance healthcare and life science customers by providing: insights based on the voice of the patient or resident; facial features diagnosed from facial recognition technology or software; social determinants of health; clinical trial candidates; new product development acceleration; predictive health outcomes; provider and integrated health network analytics; payor per-member per-month patient, resident and caregiver insights; longitudinal insights on patient, resident and caregiver stress, depression and anxiety mitigation tools and strategies; caregiver well-being analysis; predictive patient and resident medication compliance and persistency challenges; and predictive cognitive stability or decline insights. The AI system also performs speech assessment of the patient or resident or caregiver through voice biomarker or facial recognition AI and ML, including assessments of anxiety and stress, depression and mood, health and wellness and vocal energy, as well as assessment of the patient or resident for mild cognitive impairment, Alzheimer's disease and dementia.

In embodiments, a user may augment their system according to different computer programs. This may be accomplished through a.) an app store which may include apps providing health and wellness tools, medication and other reminders, virtual social therapy, health and wellness education, event triggered education and research, polls, surveys and caregiver support, daily affirmations, music streaming, safety and companionship communications, physical therapy instructions, caregiver resilience training, mental and telehealth communications, music and reading therapy, and other value-added partners; b.) speech assessment through voice biomarker artificial intelligence and machine learning, which may include analysis and evaluation of anxiety and stress levels for the patient and caregiver, depression and mood levels for the patient and caregiver, health and wellness evaluations for the patient and caregiver, mild cognitive impairment evaluation for the patient, and Alzheimer's and dementia evaluation for the patient; c.) AI and ML enhanced reporting, member insights database which may include patient reported outcomes, leading health indicators, data from the caregiver and health care team portal, both syndicated and custom research data, reminder acknowledgements, and social determinants of health. Based upon all of the foregoing, aspects of the disclosure analyze a vast amount of data in order to provide evaluations of the patient and caregivers and recommendations for modifying and hopefully improving the protocols for communicating with the patient and/or the caregivers; and d.) facial recognition through artificial intelligence and machine learning, which may include analysis and evaluation of anxiety and stress levels for the patient and caregiver, depression and mood levels for the patient and caregiver, health and wellness evaluations for the patient and caregiver, mild cognitive impairment evaluation for the patient, and Alzheimer's and dementia evaluation for the patient;

The components of the AI and ML platform incorporating may include a.) structured data sources including structured data from databases, speech data, electronic health records data, census data, structured database data, customer relationship management (“CRM”) data and enterprise resource planning (“ERP”) data; b.) semi-structured data sources including diagnostic tools, lab results, and internet of things, which provide Javascript object notation (“JSON”) data, extendible markup language (“XML”) data, comma-separated values (“CSV”) data, and hyper-text markup language (“html”) data; and c.) unstructured data including unstructured data from a member community ecosystem database, social media databases, and data from wearable databases such as watches and other health related wearables, which may include videos, audio files, portable document format (“pdf”) data, sensor data, and social media data. The digital health companion and information gathered then undergoes a data ingestion process for the batch data, the scheduled data, and the real-time streaming data, which is then extracted and loaded into the raw or landing data storage database, which communicates with both data analytical sandboxes which may include a data discovery sandbox, an exploratory analysis sandbox, and a predictive modelling sandbox. The raw or landing storage database is also in communication with a transforming module which may perform both batch data processing and real-time processing, resulting in processed data which is communicated to the analytical sandboxes, such as the data discovery sandbox, the exploratory analysis sandbox, and the predictive modelling sandbox. The processed data may also be communicated to the data consumption module which may include business intelligence (“BI”) and related analytics, which may be also known as a decision support system (“DSS”), data reporting, a data warehouse, real-time alerts, and search and query capabilities. The foregoing system provides data security, data governance and data monitoring. As will be understood, conversion technologies for accessing and processing data from different formats and sources are included.

The data sources, which are gathered and transmitted to the AI engine, include cognitive and behavioral health assessment data, digital health companion activity data, electronic health records, lab results, diagnostic tools, wearable health trackers, fall detection and other home safety devices, remote patient monitoring tools, pharmacy benefit manager (“PBM”) data and pharmacy data. The data is all transferred to a data lake central repository for storing all healthcare-related raw data. The AI artificial intelligence engine analyzes the data for predictive analytics, health outcomes, disease patterns and potential modifications to the dynamic care plan for the digital health companion. The AI engine output is transmitted to stakeholder applications as necessary. These can include, but not be limited to, discrete devices, assisted living communities, memory care communities and home healthcare patients and caregivers, subscribers or payers, pharmacy benefit managers, associations and foundations involved in healthcare, pharmaceutical companies, biotech companies, medical device manufacturers and medical device users and their caregivers, healthcare providers and related entities, and other strategic partners involved with providing and/or gathering data or research.

Further, aspects of the disclosure include compiling a member insights database which may comprise the digital monitors user's demographics, responses and activities, plus third party published and unpublished data, reports and articles, syndicated and custom research, social determinants of health, cognitive/behavioral health assessments, partner app utilization reports, self-reported health and wellness assessments and tele-health integration reports.

Herein is presented a method of communication between persons which comprises communication between at least two entities while utilizing an artificial intelligence system. Aspects of the present disclosure include the use of a digital health companion device capable of implementing a dynamic care plan which monitors, stores and analyzes activities on the digital home companion. In some embodiments, a companion app allows caregivers and any other second person and/or third person to receive alerts and data from the digital health companion and modify the content and tools accessible to the first person on the digital health companion. The AI system may provide for real-time monitoring to develop a dynamic care plan and can monitor and analyze the first person's activities on the digital health companion, and recommend or implement modifications to the content and tools accessible on the digital health companion. The dynamic care plan may be enhanced by AI to analyze patient activity and modify the care plan.

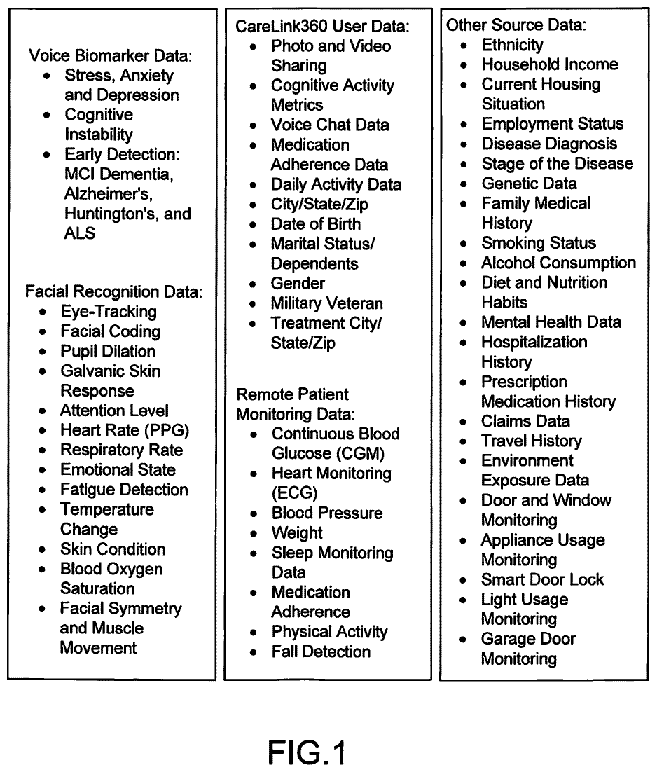

FIG. 1 identifies the data inputs for the artificial intelligence system. The listed data inputs are merely one embodiment of the possibilities for databases that may be accessed. The figure describes the extend of the database footprint and members that may have input into the AI system. After processing the databases illustrated in FIG. 1, different individuals and communities may utilize the data, upon proper access. This may include customers ranging from single community settings to multi-community settings, such as senior living communities, assisted living facilities, memory care communities, continuing care retirement communities, and in-home care agencies and individuals. Additional customers may include caregivers, paying subscribers, and pharmaceutical-related entities. Access to the data may be obtained through paid subscription to an app store. Other apps may also be linked to the AI system such as health and wellness tools, health and wellness education, medication and other reminders, event triggered education and research, virtual social therapy, polls or surveys and caregiver support, daily affirmations, caregiver resilience training, music streaming, mental and telehealth, safety and companionship, music and reading therapy and physical therapy.

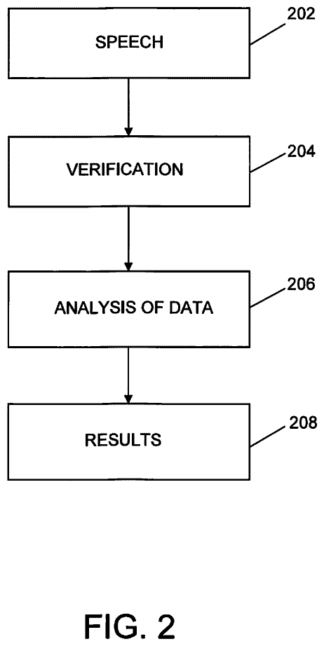

FIG. 2 illustrates the inclusion of speech recognition capabilities into the AI system. At 202, FIG. 2 shows speech being accepted from a patient and/or caregiver. At 204, the data from the speech is input into a processor and verified. The processor may be part of the AI system or may be a separate computing device. At 206, the data may be analyzed for patterns and traits that may indicate patient symptoms, emotions or other conditions. This speech assessment may be accomplished through voice biomarker artificial intelligence and machine learning. Results, at 208, that are generated may include the assessing the anxiety and stress of the patient and caregiver, the mood and level of depression of the patient and caregiver, the health and wellness of the patient and caregiver, the vocal energy of the patient and caregiver, any mild cognitive impairment of the patient, and any Alzheimer's disease or dementia of the patient. The input mechanism for the analysis performed is accepted when access is granted. Insights, using artificial intelligence and machine learning may be provided including susceptibility to chronic, terminal, and rare diseases. Healthcare may be advanced through evaluation of a patient voice over time to track progress and/or decline. Outcomes of evaluation may include qualification of health clinical trial candidates, new product administration, predictive health outcomes, provider and integrated health network analytics, payor per-member per-month patient and caregiver insights, longitudinal insights on resident, patient and caregiver stress, depression and anxiety mitigation tools and strategies, caregiver well-being analysis, predictive patient and resident medication compliance and persistency challenges, and predictive cognitive stability or decline insights.

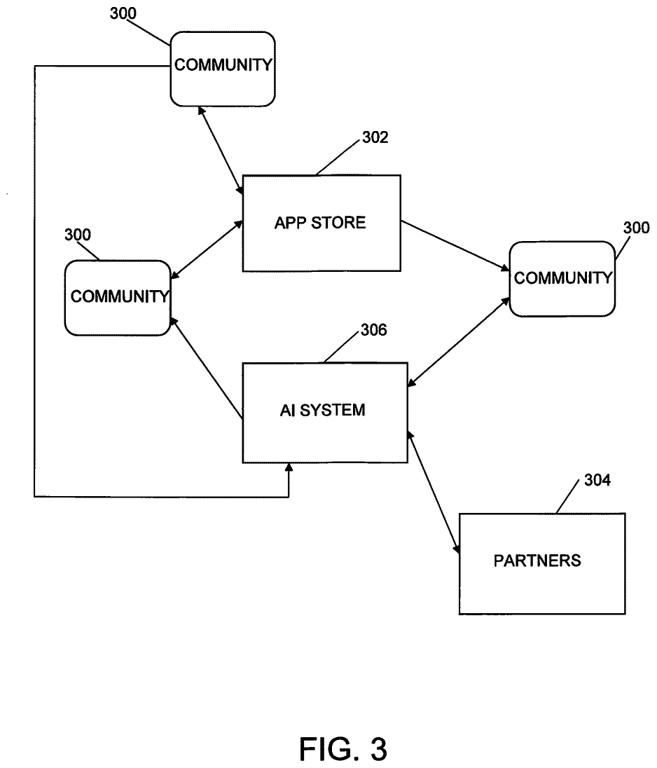

FIG. 3 is an illustration incorporating AI into the ecosystem and platform for the dynamic care plan of a digital health companion. FIG. 3 identifies a member community 300, the app store 302, partners 304, and the artificial intelligence system 306 with enhanced reporting and member insights database. The digital health companion member community includes providers, payers, pharmaceutical-related entities, assisted living communities, memory care companies and health care companies. FIG. 3 illustrates that the app store may include choices such as health and wellness tools, medication and other reminders, virtual social therapy, health and wellness education, event triggered education and research, polls and surveys and caregiver support, daily affirmations, music streaming, safety and companionship, physical therapy, caregiver resilience training, mental and telehealth, music and reading therapy, and other value-added partners. A digital health companion AI-enhanced reporting and member insights database incorporating AI may provide patient reported outcomes, leading health indicators, as well as a caregiver and health care team portal. Together, the member community 300, app store 302, partners and AI-enhanced reporting and member insights database incorporating AI 306 comprise the components to provide a dynamic care plan. As described, partners 304 may include various entities that wish to access and/or provide information from the AI system 306. Partners may provide compensation to the operators of the AI system 306 for access. In other embodiments, partners may include non-profit institutions that wish to access the processing capability of the AI system 306. Such institutions may have a waived subscription fee.

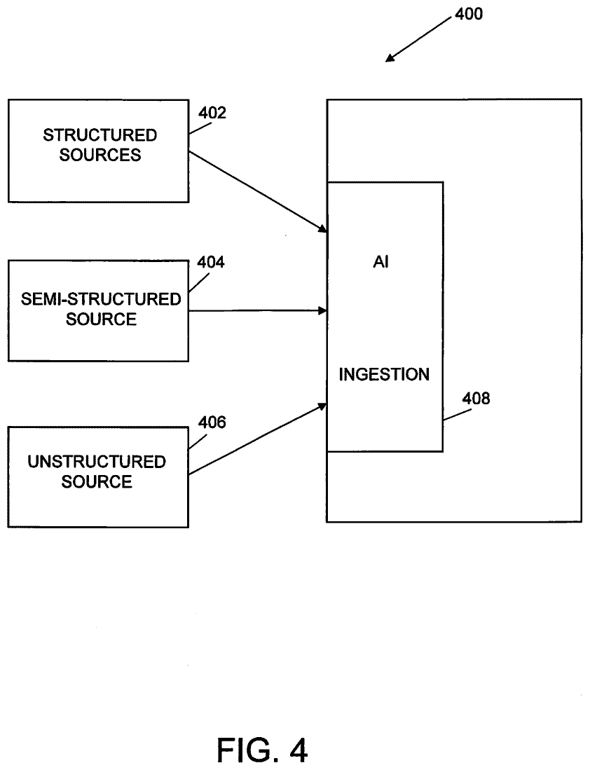

FIG. 4 is an illustration of the components of the disclosure with AI artificial intelligence and machine learning platform 400. Data sources which are accessed include structured data sources 402, semi-structured data sources 404 and unstructured data sources 406. The structured data sources may include the digital health monitor data, speech assessment data, electronic health records, wearable devices and census data. The structured data types include databases, customer relationship manager (“CRM”) data, and enterprise resource planning (“ERP”) data. The semi-structured data sources include diagnostic tools, lab results, and internet of things (“IoT”) data. The semi-structured data types include JavaScript object notation (“JSON”), extensible markup language (“XML”), comma-separated values (“CSV”), and hypertext markup language (“HTML”). The unstructured data sources include social media databases. The unstructured data types include videos, audio files, portable document format (“PDF”) files, sensor data, and social media data. FIG. 4 further illustrates that the AI system transfers all of the data gathered into a data ingestion module 408 which performs both batch and scheduled data ingestion and real-time streaming data ingestion. All of the ingested data is extracted and loaded into a raw data landing or raw data store. The raw data store sends the data to both analytical sandboxes and to data transformation module. The analytical sandboxes include data discovery, exploratory analysis and predictive modelling. The data transform module performs both batch processing and real-time processing on the data and sends the data to a processed data module. The processed data module communicates the processed data to both the analytical sandboxes, described above, and to the data consumption module. The data consumption module performs business intelligence (“BI”) analytics, reports the data, acts as a data warehouse, communicates real-time alerts to a designated community, and performs searches and queries on the data. The artificial intelligence and machine learning platform also includes a data security, governance, and monitoring module.

FIG. 4 identifies different data sources and capabilities used/created by the AI system, including tools, education, virtual social therapy (“VST”, video chat), reminders and data analytics. Non-limiting embodiments of tools may available may include brain fitness modules for simple, soothing and stimulating activities that older adults can perform easily and frustration-free with the use of one finger or stylus. Further tools may also include more traditional activities such as jigsaw puzzles, solitaire, word search and more.

Different types of prescribed activities may be included as part of the dynamic health care plan. These may include various activities such as sitting and gentle exercises for older adults; gentle yoga and breathing exercises for either sitting or standing positions; visual and audio books designed for older adults living with dementia, Alzheimer's disease or mild cognitive impairment; soothing videos of nature, animals, birds for enjoyment and relaxation; sing-along songs and other music therapy; speech therapy; and health risk and health assessment questionnaires. More complex identified problems with patients may increase the need for caregiver assistance and alert medical staff or family members of needed medical care.

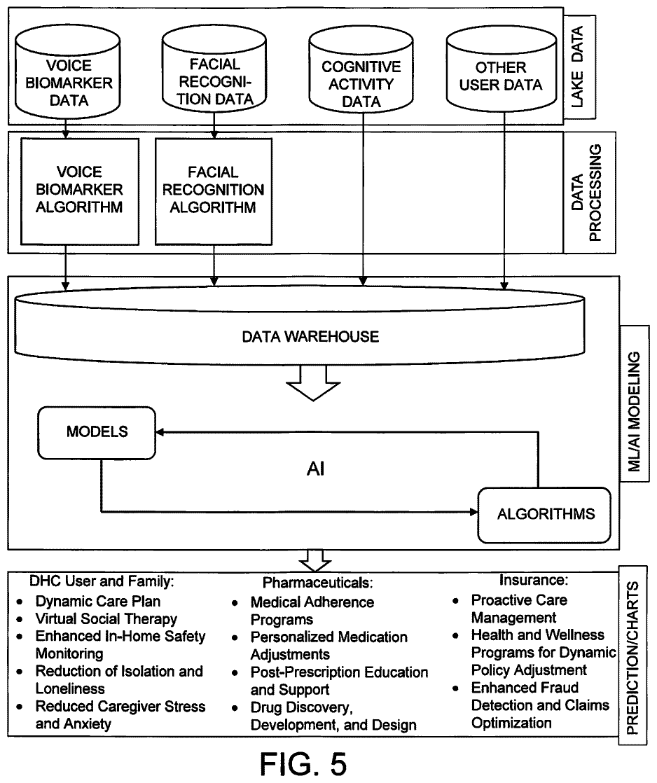

FIG. 5 is an illustration identifying data sources, a data lake and AI engine, and stakeholder applications. The AI engine gathers extensive data from varied data sources; the data is stored in a data lake and analyzed by the AI engine; and the data is then communicated with a number of stakeholder applications. The data sources identified include cognitive and behavioral assessment data, digital health companion activity data, electronic health records, lab results, diagnostic tools, wearable health trackers, fall detection and other home safety devices, remote patient monitoring tools, prescription benefit management (“PBM”) and pharmacy-related data. The AI data lake is a central depository for storing all healthcare-related raw data, and the AI artificial intelligence engine analyzes all of the data for predictive analytics, health outcomes, disease patterns and for potential modifications to a dynamic care plan. As illustrated, prediction/charts may be generated as portions of the dynamic care plan. As will be understood, each of the components within the data lake may be encrypted or locked. In other embodiments, the data may be stripped of identifying patient information to provide for “raw” data that does not have identifiers for individual patients. This data may then be processed by the AI engine. The AI engine then transmits the results of the data analysis to stakeholder applications including to members of digital health companion ecosystem, assisted living facilities, memory care facilities and home healthcare entities, subscription payers, prescription benefit management entities, associations, foundations, pharmaceutical-related entities, biotech-related entities, health care devices, health care professionals (“HCP”), health care providers and strategic partners.

The processed data usage or transfer from the stakeholder or output applications may, in one embodiment, be viewed as segmented into phases. One phase of the output may include a digital monitoring system utilizing the processed data to display or modify the dynamic care plan, the health care monitoring and predictive care, as well as the safety and security, the care coordination, the communication and connectivity and the smart home and IoT technology. In another phase, the assisted living facilities, memory care facilities and home health care entities may use the processed data to improve operational efficiency, resource management, enhanced communication, resident monitoring and safety, predict health analytics, dietary and nutrition management, security enhancements, optimized staff training and development, and recruitment and retention. In another phase of the utilization of the processed data from the AI engine may be viewed as the use by the commercial industry, government entities and foundations. In this phase, the subscription payers, pharmaceutical benefit managers, associations, foundations, pharma and biotech related entities, medical device related entities, health care providers, home care providers, and government entities, such as the World Health Organization, the National Institute of Health may use the processed data for health and wellness program utilization, drug utilization, member registry management, donation management, patient and caregiver support, clinical trial candidate recruiting, patient journey mapping, social determinants of health, patent extension opportunities, new product development acceleration, post market surveillance, management of CT data sets, global epidemic management, public health research, monitoring, and evaluation.

Different aspects of a patient or a caregiver may be tracked. Non-limiting embodiments of what may be tracked include aspects related to:

### 1. Physical Health:

Vital Signs: Blood pressure, heart rate, respiratory rate, and temperature.
Body Measurements: Height, weight, Body Mass Index (BMI), waist circumference.
Physical Fitness: Strength, flexibility, endurance, and mobility assessments.
Chronic Conditions: Presence of diseases such as diabetes, hypertension, or cardiovascular issues.

### 2. Mental Health:

Psychological Well-Being: Assessments of mood, anxiety, depression, and stress levels.
Cognitive Function: Evaluations of memory, attention, and problem-solving abilities.

### 3. Lifestyle Factors:

Diet and Nutrition: Eating habits, dietary restrictions, and nutritional intake.
Physical Activity: Levels of exercise, sedentary behavior, and active lifestyle.
Substance Use: Alcohol consumption, smoking, and drug use.

### 4. Social Determinants of Health:

Living Conditions: Housing quality, neighborhood safety, and access to resources.
Social Support: Relationships with family, friends, and community involvement.
Economic Stability: Employment status, income, and financial security.

### 5. Preventive Health Measures:

Screenings: Regular health screenings (e.g., cholesterol, cancer screenings).
Immunizations: Vaccination status and adherence to recommended immunization schedules.

### 6. Self-Reported Health:

Health Perception: Individual's assessment of their own health status.
Quality of Life: Measures of satisfaction with life and health-related quality of life.

Aspects of the disclosure allow AI assessments that will help healthcare providers develop personalized health plans, identify risk factors, and monitor changes over time.

In embodiments, the AI system provides a risk assessment that evaluates an individual's potential health risks and identifies areas for improvement. Individual aspects that may be tracked include data obtained directly from a patient or in a database. These may include, but are not limited to:

### 1. Demographic Information:

Age, gender, ethnicity, educational level, and occupation.

### 2. Medical History:

Personal and family medical history, including chronic conditions and past surgeries.

### 3. Lifestyle Factors:

Diet: Eating habits, nutritional intake, and dietary restrictions.
Physical Activity: Level of exercise, frequency, and types of physical activities.
Substance Use: Alcohol consumption, tobacco use, and recreational drug use.
Sleep Patterns: Quality and duration of sleep.

### 4. Vital Signs:

Blood pressure, heart rate, respiratory rate, and temperature.

### 5. Body Measurements:

Height, weight, Body Mass Index (BMI), and waist circumference.

### 6. Mental Health:

Assessments of stress, anxiety, depression, and overall mental well-being.

### 7. Screening Results:

Results from relevant screenings, such as cholesterol levels, blood glucose levels, and cancer screenings.

### 8. Preventive Health Behaviors:

Participation in preventive measures, such as vaccinations and regular health check-ups.

### 9. Social Determinants of Health:

Factors including socioeconomic status, education level, and access to healthcare resources.

### 10. Health Perception:

Individual's self-assessment of their health status and quality of life.

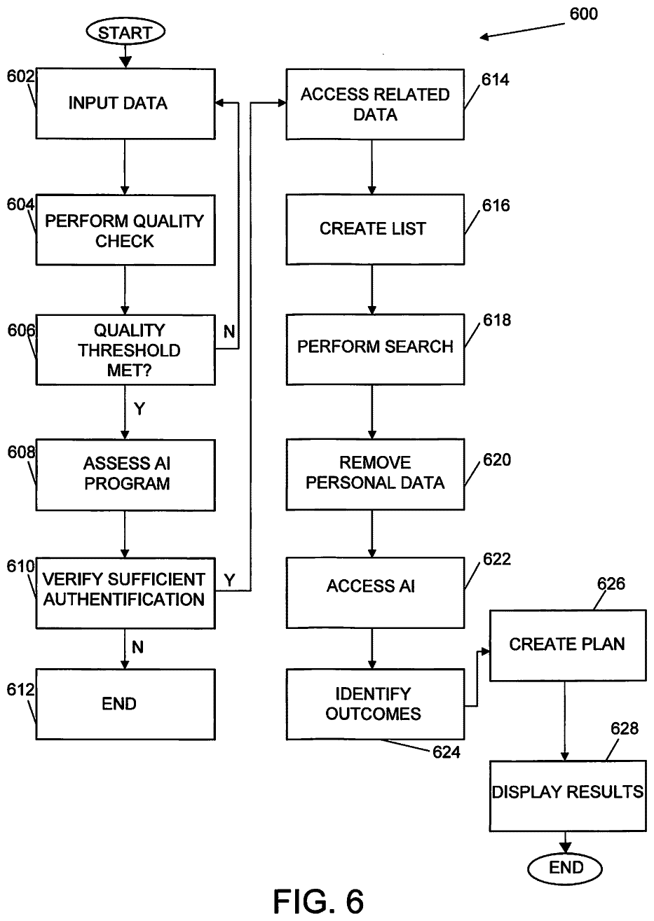

Referring to FIG. 6, a method 600 to produce a health care plan for an individual is illustrated. The method may comprise, at 602, inputting data related to an individual, the data related to at least one health care statistic for the individual. The method may further comprise, at 604, performing a quality check of the input data related to the individual. The method may further comprise, at 606, looping back to the inputting data related to the individual when the quality check indicates that a threshold amount of data of sufficient quality has not been satisfied. The method may further comprise, at 608, accessing an independent artificial intelligence program. The method may further comprise, at 610, performing an authentication check of the accessing of the artificial intelligence program to verify that the accessing possesses sufficient authentication for artificial intelligence access. The method may further comprise, at 612, ending the method when the authentication check to the artificial intelligence fails. The method may further comprise, at 614, accessing at least one database that contains data related to healthcare patients other than the individual. The method may further comprise, at 616, creating a list of data to be searched with the at least one database based upon input data from the individual. The method may further comprise, at 618, searching the at least one database based upon the list of data to be searched to find similar individuals having data withing the at least one database and retaining the matching data. The method may further comprise, at 620, removing individual personal identifying information from the matching data. The method may further comprise, at 622, using the artificial intelligence program to sort and analyze the matching data to identify a current healthcare status for the individual. The method may further comprise, at 624, using the current healthcare status for the individual and the matching data, identifying likely future healthcare outcomes for the individual. The method may further comprise, at 626, creating the healthcare plan for the individual based on the likely future healthcare outcomes for the individual. The method may further comprise, at 628, at least one of displaying and saving the health care plan for the individual.

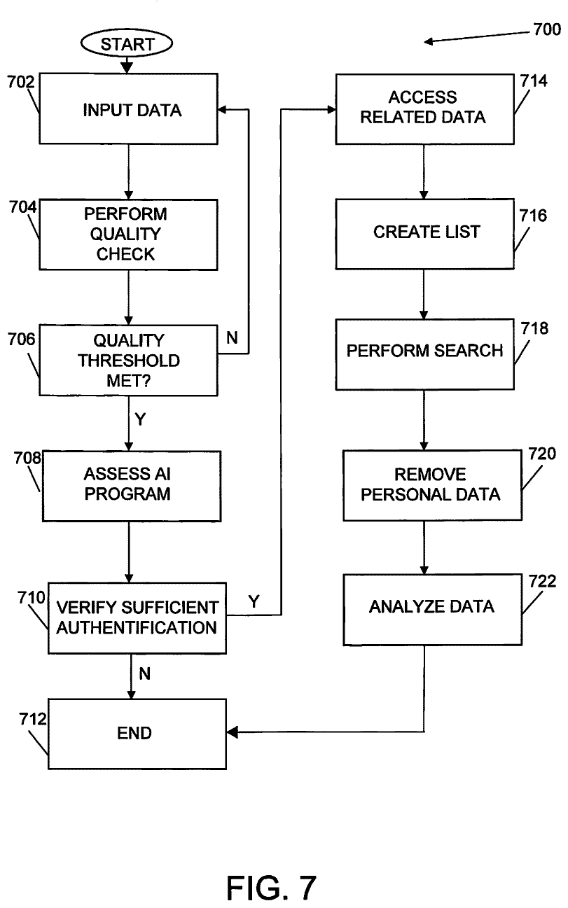

Referring to FIG. 7, a second method 700 is disclosed. The method determines a health care status of at least one individual. The method comprises, at 702, inputting data related to the at least one individual, the data related to at least one health care statistic for the individual. The method further comprises, at 704, performing a quality check of the input data related to the individual. The method further comprises, at 706, looping back to the inputting data related to the individual when the quality check indicates that a threshold amount of data of sufficient quality has not been satisfied. The method further comprises, at 708, accessing an independent artificial intelligence program. The method further comprises, at 710, performing an authentication check of the accessing of the artificial intelligence program to verify that the accessing possesses sufficient authentication for artificial intelligence access. The method further comprises, at 712, ending the method when the authentication check to the artificial intelligence fails. The method further comprises, at 714, accessing at least one database that contains data related to healthcare patients other than the individual. The method further comprises, at 716, creating a list of data to be searched with the at least one database based upon input data from the individual. The method further comprises, at 718, searching the at least one database based upon the list of data to be searched to find similar individuals having data withing the at least one database and retaining the matching data. The method further comprises, at 720, removing individual personal identifying information from the matching data. The method further comprises, at 722, using the artificial intelligence program to sort and analyze the matching data to identify a current healthcare status for the individual.

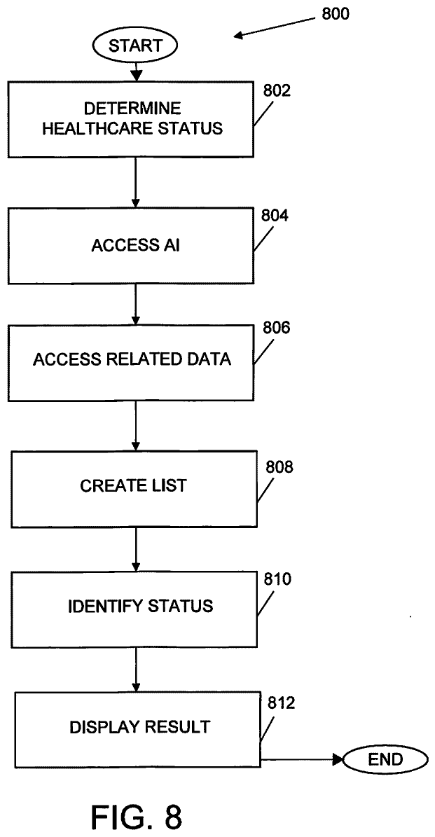

Referring to FIG. 8, another example embodiment of the disclosure is presented. The method 800 may comprise determining a health care status of at least one individual. The method 802 may further comprise accessing an independent artificial intelligence program. The method may further comprise, at 804, accessing at least one database that contains data related to healthcare patients other than the individual. The method may further comprise, at 806, selecting health care data for the at least one individual. The method may further comprise, at 808, creating a list of individuals with similar health care data to the at least one individual. The method may further comprise, at 810, using the artificial intelligence program to sort and analyze the matching data to identify a current healthcare status for the individual to achieve a result. The method may further comprise, at 812, at least one of displaying and printing the result.

### Dedicated Device

In some aspects, data input may be performed on a dedicated device. The dedicated device may provide for authentication of a user and input of various forms of data for tracking. The authentication may occur through voice recognition, visual recognition, biomarker recognition, password or other protected input. The dedicated device may have a non-intrusive presence and may be accessed by a user at will. The device may enter a low-power mode when not in use to limit the overall electrical usage. The device may be equipped with software that has adaptive features and functions that may be loaded on, over time, as needed. Thus, the dedicated device may be internet compatible, cell phone compatible, mainframe compatible and/or any combination described above. Aspects of the software may be used to create a safe and secure emotional companion device for persons, such as the elderly or children, when needed. Oversight of the dedicated device may be accomplished/performed by an administrator.

In aspects of the disclosure, using individuals can rely on an ongoing and continued dialogue with the dedicated device in an easy and automatic way. Aspects of the device may present a view on screen or by alternative means and methods of the state of user, and options that are available to the user. In one non-limiting embodiment, an adapted interface might provide pictures of family members of the user.

The dedicated device solicits engagement by the user and provides helpful information, simplified access to communication, reminders, schedules, healthcare monitoring and management, and is updated automatically without intervention by the user. The dedicated device is the input device to a method to manage communication, monitor, articulate, and verify the protected person's state, and adapt or promote actions to improve emotional valence and social interaction, to reduce isolation, stress, and loneliness, to reduce complexity, and for the best health outcomes. These data streams may be fed into the artificial intelligence system where processing occurs producing a dedicated and dynamic care plan for the user. The features of the dynamic care plan created by the artificial intelligence system may be approved by the administrator and/or may be automatically implemented. In the case of automatic implementation, the care plan may require altering medical care, such as changing prescriptive medications. Other options may include creating an appointment to see a dedicated medical professional if symptoms warrant. In some embodiments, the medical appointments may be performed directly on the dedicated device, thereby limiting the overall cost and complications of travel for the user.

### Communication

The devices and methods described herein assist and/or allow for communication between two or more people. The artificial intelligence system that obtains data may select data from a variety of sources. Various types of data may be used. Oral and/or video communication may be used. It is understood; however, that communication can occur in many forms. It is understood that communication can occur with voice alone, or by text, with alternative media, a robot or similar device, with the assistance of sensors, actuators, or through specialized communications techniques for example, symbols, Braille or similar tactile means, specialized methods including speech to text to graphically generated sign language, through a relay operator or person acting in that capacity, captioning, or sign language on a video display.

It is also understood that a TDD system can be used for data input that is ultimately analyzed by the artificial intelligence system. TDD systems are also known as teletypewriter, TTY, textphone, and minicom, MCM, Def-tone, DTS, each of these terms are used interchangeably herein.

Communication to and from the AI system can also be through touch. This is particularly useful for some protected people for whom traditional communication by voice or text is intimidating. In one configuration the AI system allows a person to select an image using a touch screen menu to convey thoughts or emotions. The image may be directly communicated to the recipient it may also be converted to another form of communication including voice or text. For instance, a patient might hear their daughter's voice and select an emoji to convey a sentiment or message, on the touch screen. Upon the selection of that image, the device can be configured to say to the daughter, “I am safe and happy.” In some embodiments this voice is a computer-generated voice. In other embodiments, the voice is prerecorded by the protected person. In still other embodiments, the device is configured to synthesize a voice to sound like the protected person having derived the protected person's voice from collected recordings.

### Electronic Means or Electronic Communication

The terms “electronic means” or “electronic communication” include end to end wired, wireless, radio transmission, and others, inclusive of campus environments such as a closed system (i.e. hospitals), including specialized means such as inductive coupling (e.g. methods of data transfer between implanted medical devices and external systems), near field communications in devices of close proximity (NFC), optical, infrared, scanners, acoustic transmission of data gathered by microphones, and many other means and methods in use to enable connectivity for devices and services. The field of networking is experiencing tremendous growth and diversification, with useful implementations for all aspects of connectivity including traditional wired and wireless communications infrastructure, with additions for simple and secure networking as well as self-configuring ad hoc networking. Over time, some are more abundant and preferred in a given environment such as a medical environment, while others are prevalent because of simplicity, cost, size, safety, embedded infrastructure, practical nature of the method of transmission, etc. Aspects of the disclosure may use the existing internet worldwide communication infrastructure, wired and wireless, as well as campus systems typically found at hospitals, schools, in homes, and elsewhere. Device to device communications are contemplated in this disclosure.

Aspects of the disclosure provide for preparation of a health care action plan for patients that is reliable.

Aspects of the disclosure provide for quick assessment of a patient's data and to be able to easily add proprietary databases into an analysis system that will summarize and predict patient outcomes.

Aspects of the disclosure provide for processing patient data in a cost-effective way to minimize medical professional resources while providing an accurate assessment of patient condition.

Aspects of the disclosure provide for tracking a caregiver's health in addition to the patient to identify and/or prevent medical complications from occurring in others aside from the patient.

Aspects of the disclosure provide for prescreening of patients for medical tests to simplify the administration of medical tests and costs.

The devices and methods described herein assist and/or allow for communication between two or more people. One of the people may be a medically-supervised or protected-person. Data access to computer records may be shared among all users or may be restricted to specific individuals needing data, such as a medical professional. Use of data by non-medically trained individuals is also contemplated, wherein a family member of a user may have access to data. Such access may allow for financial planning, medical planning and estate planning of individual(s) being tracked.

In some situations, embodiments of the disclosure relate to elderly individuals or the aging. In some further instances, the individual has a diminished capacity. In some situations, a physician, hospital or clinic has determined that the person lacks capacity to make decisions. In some situations, a court has determined that the person's mental functions are diminished or impaired. In some situations, the court determination is statutory; for instance, a parent or guardian has legal control for their child or ward, at least until the protected person is of legal age. In many situations, the protected person has knowingly volunteered to defer control of their communication to another person for any one of a variety of personal reasons. The status of the protected person may be either permanent or temporary. Other situations are possible wherein the person is not elderly but wishes to track medical progress or create a dynamic care plan do to existing physical conditions, regardless of the presence of any physical impairment. In other more extreme conditions degenerative diseases may be present where tracking of physical status would provide a benefit.

All illustrations of the drawings are for the purpose of describing specific embodiments of the invention and are not intended to limit the scope of the disclosure.

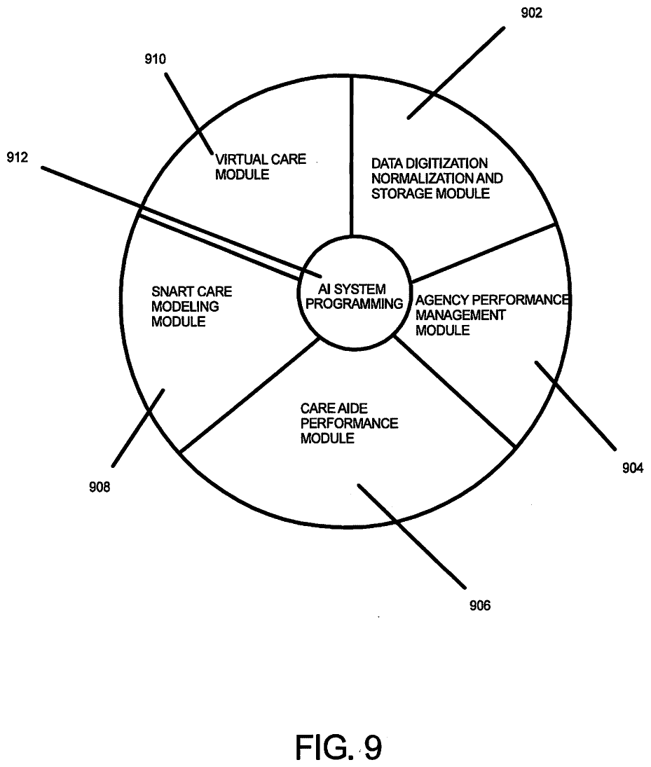

Referring to FIG. 9, a diagram illustrating different modules of an ecosystem used in the process of administering medical care is illustrated. Individual modules include a data digitization, normalization and storage module 902 and an agency performance management module 904. Individual modules also include a care aide performance module 906, and a smart care modeling module 908. A virtual care module 910 is also provided.

Each of the modules 902, 904, 906, 908, 910 are configured to interact with programming 912. The programming 912, in one example embodiment, may be a previously described artificial intelligence program. In embodiments, the programming 912 may be configured to interface with a storage system to allow for efficient storage of data. As will be understood, the storage system may have an incorporated or external security protection program to prevent unauthorized access to data stored relating to the modules 902, 904, 906, 908, 910 and programming 912.

In embodiments, the programming 912 and the individual modules 902, 904, 906, 908, 910 may reside on a single computing apparatus. The computing apparatus may be, in one non-limiting embodiment, a computer server may be used. In some embodiments, a cloud-computing arrangement may be used. Such cloud-computing arrangements may include a distributed computing capability where portions of computing functions may be performed on various computer servers.

Referring to FIG. 9, the module 902 is configured to accept and, if necessary, convert different forms of data required to be input. The data digitization normalization and storage module 902 allows different formats of data to be input into the module 902 and stored for usage. The module 902 is provided to convert different formats of data, such as PDF format materials and places the data in a structured manner. The module 902 may be configured to auto-extract data from the different sources. Such sources shall be, for example visit notes from doctors or other medical professionals. The data may be scanned through various means including mobile phone, computer and copier as non-limiting embodiments.

After input of the data, a process of data normalization is performed. As defined herein, data normalization is the process of structuring a database by eliminating redundancy and organizing data efficiently. In embodiments, the data integrity is ensured. In embodiments, the data normalization standardizes the data across various fields that may be stored.

In aspects of the disclosure, the normalization process accepts data and then checks existing databases to see if the data is already present within the database. If the data is not incorporated, the data may be incorporated. If the information is a copy of existing data, then the data may be chosen to not be added. In some instances, the data may be modified in some fashion. The normalization process then modifies the record according to set parameters by an algorithm.

Data normalization may encompass several methodologies to ensure that information is efficiently structured, redundancies are minimized, and data integrity is preserved. The most foundational type is First Normal Form (1NF), which requires that every field in a database table contains only atomic—indivisible—values, and that each record is unique. This means repeating groups and arrays within records are eliminated, establishing the groundwork for systematic organization. By enforcing this rule, First Normal Form transforms raw, often unorganized data, into a clear and accessible structure suitable for further refinement.

Building upon the foundation of 1NF, Second Normal Form (2NF) and Third Normal Form (3NF) introduce more sophisticated normalization techniques aimed at reducing data duplication and enhancing relational clarity. Second Normal Form is achieved when data is already in 1NF, and every non-primary-key attribute is fully dependent on the table's primary key, eliminating partial dependencies. Third Normal Form, meanwhile, takes the process further by ensuring that all columns are only dependent on the primary key, not on other non-key attributes, thereby removing transitive dependencies. This layered approach leads to databases that are not only efficient but also easier to maintain and update as organizational needs evolve.

Beyond these conventional forms, advanced normalization types such as Boyce-Codd Normal Form (BCNF), Fourth Normal Form (4NF), and Fifth Normal Form (5NF) address increasingly complex relationships and anomalies that may arise in large-scale or highly interconnected datasets. BCNF tightens the rules surrounding functional dependencies, while 4NF focuses on eliminating multi-valued dependencies, which can occur when a table contains two or more independent and multivalued facts about an entity. Fifth Normal Form, the most rigorous, handles cases where information can be reconstructed from smaller pieces of data, allowing for maximal flexibility without redundancy. Together, these types of normalization provide a spectrum of tools for modeling data in ways that support accuracy, consistency, and scalability in modern data systems.

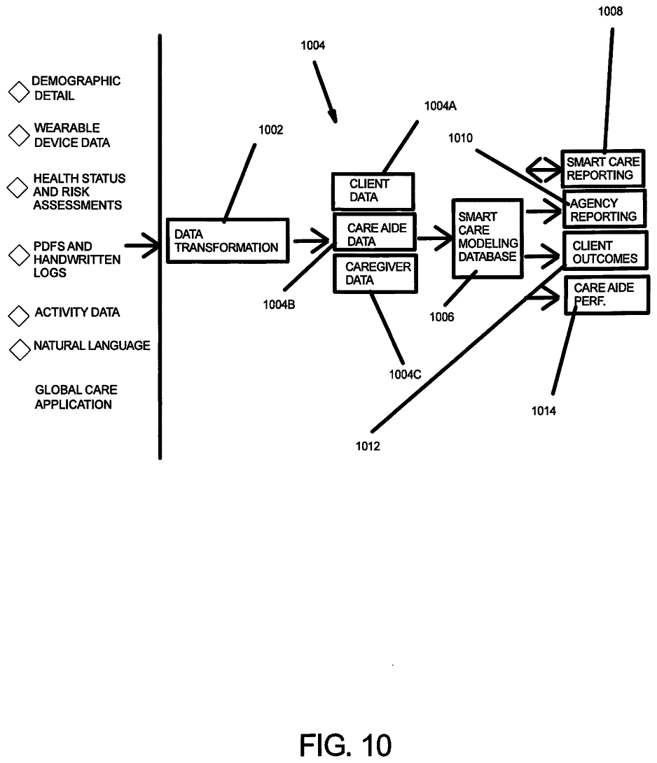

Referring to FIG. 10, further aspects of the data digitization, normalization and storage module 902 are illustrated. At the left, different types of structure, semi-structured, or unstructured data sources may be accessed, obtaining data through various means such as sensor monitoring, computer hardware access storage, health records, wearable devices, health status and risk assessments previously made, pdf files and/or handwritten logs, activity data, natural language databases or other applications. The data disclosed at the left of FIG. 10, may be transformed, at 1002 to be placed in a database 1004. The database 1004 may be a computer storage device, such as a solid-state device, a computer server, a hard disk device or a cloud storage device as non-limiting embodiments. The data that has been transformed at 1002 may be sorted into various fields or database sections. These sections may include client data 1004A, client aide data 1004B and caregiver data 1004C.

The data from the database 1004 may then be exported to a smart care modeling database 1006. The smart care modeling database 1006 may be used to allow for alteration of data for calculation purposes. To this end, the use of the smart care modeling database 1006 does not contaminate or alter the data contained within the database 1004.

The smart care modeling database 1006 may have calculation capabilities itself or be accessed by other computing apparatus to produce usable data for individuals. The data may include, for example, smart care reporting 1008, agency reporting 1010, client outcomes 1012 and care aid performance 1014. As will be understood, checks on care aid performance are conventionally done by supervisors directly with individual employees. Such evaluations are not based on data. The smart care modeling database 1006 provides a more logical and data driven approach that recognizes not only unsuccessful outcomes for patients but also successful outcomes. As will be further understood, some care aid performance may vary across different types of care. For example, one caregiver may have very successful outcomes with advanced aged patients, but less successful outcomes with younger patients. Such a system can recognize and sort caregivers according to different data givers. The results can then be used to enhance overall effectiveness on a patient facility care basis.

Trends may be developed over time for client outcomes, 1012, agency reporting 1010, smart care reporting 1008 and care aid performance 1014. These trends may be compared, for example, to national statistics to develop a more accurate picture of overall healthcare outcomes for a particular facility or geographic area. As will be further observed, more positive outcomes for a particular facility may provide other advantages, when used with other services. As a non-limiting example, access to the overall architecture by an insurance provider may indicate that a specific geographic area or facility has superior service compared to an industry average, therefore qualifying the area or facility for a reduction in insurance rates. In further embodiments, particularly poor results may be observed in care aid performance with an individual prompting quick action by supervisory staff to intervene and prevent substandard results.

As will be further understood, data may be shared on a larger geographic area or a national level. In such embodiments, verification of employment data may be obtained with the overall results obtainable by hiring facilities. Such embodiments prevent unauthorized access of care individuals to areas that they are not trained or certified for. On a further evaluation level, small organizations or facilities that may not receive recognition may be successfully compared to larger and better funded organizations. In such evaluations, cost of care per patient may be compared between facilities that would not normally be compared. Such evaluations may be provided to prospective patients to show that outcomes may be superior in one aspect, such as patient outcomes or financial considerations. If novel approaches are being used at certain facilities, such as a clinical trials, such results may be indicated in the client outcomes portion, allowing for early identification of superior healthcare. Access to the smart care modeling database 1006 and smart care reporting 1008, agency reporting 1010, client outcomes 1012 and care aid performance 1014 may be provided to consumers to allow for proper choice of desired outcomes. As will be understood, data may be stripped of personal information that should not be disclosed to the general public preventing violation of privacy requirements.

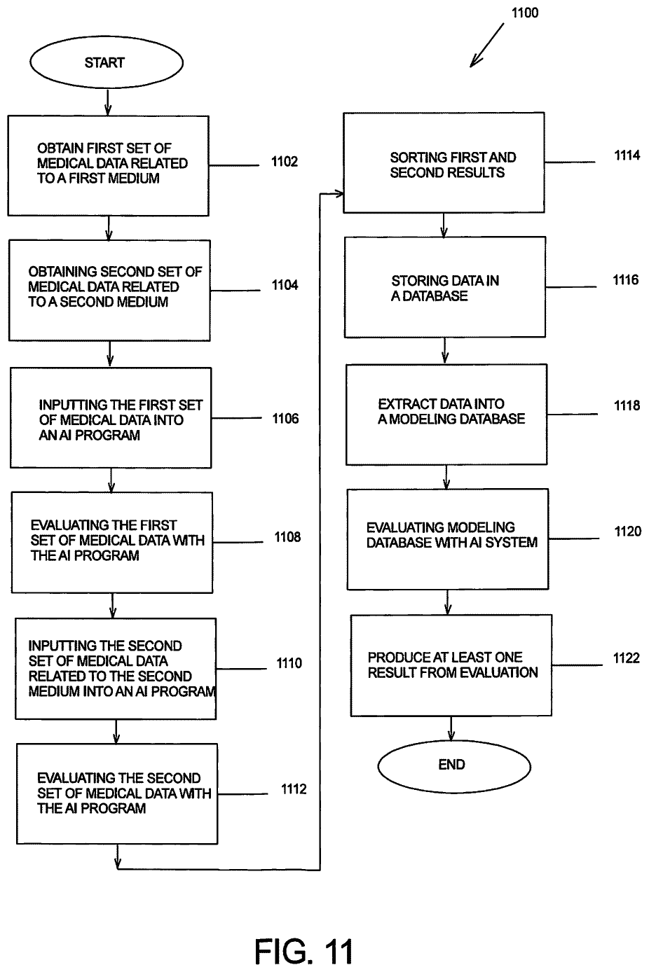

Referring to FIG. 11, one example embodiment of a method 1100 of processing medical data is disclosed. The method 1100 may comprise, at 1102, obtaining a first set of medical data related to a first medium. The method may further comprise, at 1104, obtaining a second set of medical data related to a second medium. The method may further comprise, at 1106, inputting the first set of medical data into an artificial intelligence program. The method may further comprise, at 1108, evaluating the first set of medical data with the artificial intelligence program to produce first results. The method may further comprise, at 1110, inputting the second set of medical data into the artificial intelligence program. The method may further comprise, at 1112, evaluating the second set of medical data into the artificial intelligence program to produce second results. The method may further comprise, at 1114, sorting the first results and the second results into database categories. The method may further comprise, at 1116, storing the first results and the second results into a database. The method may further comprise, at 1118, extracting data from the database into a modeling database. The method may further comprise, at 1120, evaluating the modeling database with the artificial intelligence system to produce evaluated results. The method may further comprise, at 1122, producing at least one report with the evaluated results. As will be understood, results may be printed, saved to a non-volatile, transmitted over the internet or displayed, as non-limiting embodiments. Evaluations may be conducted through artificial intelligence systems or through nodal analysis systems. Data may be sorted, for example, by the artificial intelligence system to delete repetitive data or eliminate erroneous data. Quality systems may be used to ensure that data obtained may be normalized and checked prior to use and/or storage.

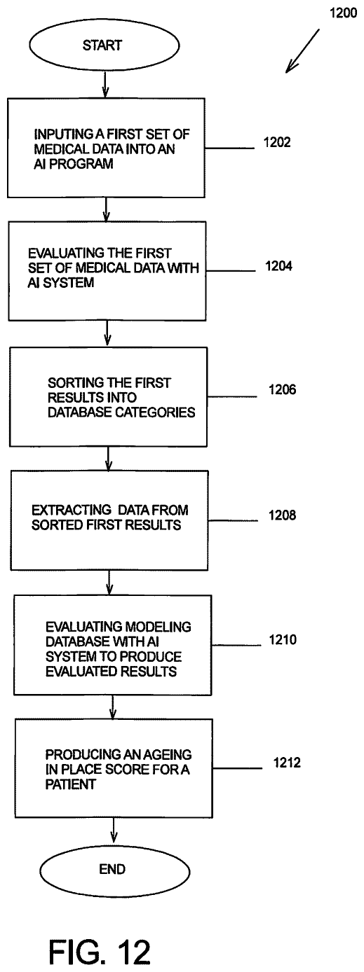

A further embodiment of a method is also disclosed. Referring to FIG. 12, a method 1200 of processing health, wellness, and medical data is disclosed. The method may comprise, at 1202, inputting a first set of health, wellness, and medical data into an artificial intelligence program. The method may further comprise, at 1204, evaluating the first set of health, wellness, and medical data with the artificial intelligence program to produce first results. The method may further comprise, at 1206, sorting the first results into database categories. The method may further comprise, at 1208, extracting data from the sorted first results into a modeling database. The method may further comprise, at 1210, evaluating the modeling database with the artificial intelligence system to produce evaluated results. The method may further comprise, at 1212, producing an aging in place score for a patient. As will be understood, results may be printed, saved to a non-volatile, transmitted over the internet or displayed, as non-limiting embodiments. Evaluations may be conducted through artificial intelligence systems or through nodal analysis systems. Data may be sorted, for example, by the artificial intelligence system to delete repetitive data or eliminate erroneous data. Quality systems may be used to ensure that data obtained may be normalized and checked prior to use and/or storage.

In embodiments, individuals, such as patients or clients, may create a profile and upload data for processing. In some embodiments, pre-defined input screens may ask questions related to general health, mental health, basic physical traits (such as weight) as well as other environmental factors. Environmental factors may include, in non-limiting embodiments, nutritional habits, tobacco use, alcohol use, sleep habits and patterns. Mental aspects of health may also be queried. Feelings of isolation and loneliness, feelings of depression, and energy levels may be input. This data may then be evaluated through the artificial intelligence system to develop, for example, an aging in place score indicating the relative health of the individual compared to other patients. This score may be updated as time progresses by further processing that occurs at a future time. Thus, an individual may enter data on a periodic basis and evaluations may be accomplished. Results may be generated and distributed, in some embodiments, to doctors, medical care professionals and family members. Suggested treatments to medical service providers may also be generated for the patient in order to improve general health.

Aspects of the disclosure provide a data accumulation system that will take various forms of medical records and store the data contained therein.

Aspects of the disclosure provide a data accumulation system that will provide for long-term safe and secure storage of such data.

Aspects of the disclosure provide for a system to convert the various forms of data into a structured format or database.

Aspects of the disclosure provide for making the structured format or database searchable by medical professionals.

Aspects of the disclosure provide data driven decisions for medical care that can anticipate or diagnose symptoms a patient may have that is outside of the experience of the medical professional.

Aspects of the disclosure provide for a system that will identify underperformance in various medical conditions compared to a norm and determine if the underperformance is related to a lack of care aide performance.

Aspects of the disclosure provide for a system that will identify overperformance in various medical conditions compared to a norm and determine when the overperformance is related to superior care aide performance.

Aspects of the disclosure provide a system that provides detection of patient health decline that is more independent from medical professionals, thus allowing newer medical professionals to ascertain changes in patient status in a quick and timely manner. As will be understood, other forms of data may also be input into the system, such as x-rays, photographs, charts and lab results. These forms of data may also be evaluated through use of the artificial intelligence system.

In one example embodiment, a method of processing medical data is disclosed. The method may comprise obtaining a first set of health, wellness and medical data related to a first medium. The method may further comprise obtaining a second set of health, wellness and medical data related to a second medium. The method may further comprise inputting the first set of health, wellness and medical data into an artificial intelligence program. The method may further comprise evaluating the first set of health, wellness and medical data with the artificial intelligence program to produce first results. The method may further comprise inputting the second set of health, wellness and medical data into the artificial intelligence program. The method may further comprise evaluating the second set of health, wellness and medical data into the artificial intelligence program to produce second results. The method may further comprise sorting the first results and the second results into database categories. The method may further comprise storing the first results and the second results into a database. The method may further comprise extracting data from the database into a modeling database. The method may further comprise evaluating the modeling database with the artificial intelligence system to produce evaluated results. The method may further comprise producing at least one report with the evaluated results.

In another example embodiment, the method may be performed wherein the first medium and the second medium are not a same medium.

In another example embodiment, the method may be performed wherein the at least one report includes data related to caregiver performance.

In another example embodiment, the method may be performed wherein the at least one report includes data related to a client health outcome.

In another example embodiment, the method may be performed wherein the at least one report includes data related to creating an agency report.

In another example embodiment, the method may further comprise displaying the at least one report with evaluated results.

In another example embodiment, the method may further comprise saving the at least one report into a non-volatile memory system.

In another example embodiment, the method may be performed wherein at least one of the first medium and the second medium is a handwritten document.

In another example embodiment, the method may be performed wherein at least one of the first medium and the second medium is one of digital information on a wearable device and data for client data.

In another example embodiment, the method may be performed wherein the at least one report indicates an aging in place value for a patient.

In another example embodiment, a method of processing health, wellness, and medical data is disclosed. The method may comprise inputting a first set of health, wellness and medical data into an artificial intelligence program. The method may further comprise evaluating the first set of health, wellness and medical data with the artificial intelligence program to produce first results. The method may further comprise sorting the first results into database categories. The method may further comprise extracting data from the sorted first results into a modeling database. The method may further comprise evaluating the modeling database with the artificial intelligence system to produce evaluated results. The method may further comprise producing an aging in place score for a patient.

In another example embodiment, the method may further comprise displaying the aging in place score for the patient.

In another example embodiment, the method may be performed wherein the evaluating the first set of medical data includes rejecting data that is duplicative of existing medical data in a database.

In another example embodiment, the method may be performed wherein the evaluating includes performing handwriting analysis of a caregiver's notes.

In another example embodiment, the method may be performed wherein the evaluating includes photos taken by the caregivers and uploaded into the caregiver notes.

In another example embodiment, the method may be performed wherein the evaluating includes updating a database with evaluated data.

In another example embodiment, the method may further comprise permitting access to proceed with the method based upon a payment system.

In another example embodiment, the method may be performed wherein the evaluating is performed on a cloud-computing arrangement.

In another example embodiment, the method may be performed wherein the inputting of the medical data is at a healthcare facility.

In another example embodiment, an article of manufacture, containing a list of instructions that is readable by a computing apparatus is disclosed. The list of instructions, at least in part, comprising: a method of processing medical data, comprising obtaining a first set of health, wellness and medical data related to a first medium. The article of manufacture having the list of instructions further has a step of obtaining a second set of health, wellness and medical data related to a second medium. The article of manufacture having the list of instructions further has a step of inputting the first set of health, wellness and medical data into an artificial intelligence program. The article of manufacture having the list of instructions further has a step of evaluating the first set of health, wellness and medical data with the artificial intelligence program to produce first results. The article of manufacture having the list of instructions further has a step of inputting the second set of health, wellness and medical data into the artificial intelligence program. The article of manufacture having the list of instructions further has a step of evaluating the health, wellness and medical data and the second set of medical data with the artificial intelligence program to produce second results. The article of manufacture having the list of instructions further has a step of sorting the first results and the second results into database categories. The article of manufacture having the list of instructions further has a step of storing the first results and the second results into a database. The article of manufacture having the list of instructions further has a step of extracting data from the database into a modeling database. The article of manufacture having the list of instructions further has a step of evaluating the modeling database with the artificial intelligence system to produce evaluated results. The article of manufacture having the list of instructions further has a step of producing at least one report with the evaluated results.

In another example embodiment, the article of manufacture may be configured wherein a form of the article of manufacture is a compact disk, a solid-state drive, a computer hard disk, and a universal serial bus device.

In one example embodiment, a method to produce a health care plan for an individual is disclosed. The method may comprise inputting data related to an individual, the data related to at least one health care statistic for the individual. The method may further comprise performing a quality check of the input data related to the individual. The method may further comprise looping back to the inputting data related to the individual when the quality check indicates that a threshold amount of data of sufficient quality has not been satisfied. The method may further comprise accessing an independent artificial intelligence program. The method may further comprise performing an authentication check of the accessing of the artificial intelligence program to verify that the accessing possesses sufficient authentication for artificial intelligence access. The method may further comprise ending the method when the authentication check to the artificial intelligence fails. The method may further comprise accessing at least one database that contains data related to healthcare patients other than the individual. The method may further comprise creating a list of data to be searched with the at least one database based upon input data from the individual. The method may further comprise searching the at least one database based upon the list of data to be searched to find similar individuals having data withing the at least one database and retaining the matching data. The method may further comprise removing individual personal identifying information from the matching data. The method may further comprise using the artificial intelligence program to sort and analyze the matching data to identify a current healthcare status for the individual. The method may further comprise using the current healthcare status and risk for the individual and the matching data, identifying likely future healthcare outcomes for the individual. The method may further comprise creating the healthcare plan for the individual based on the likely future healthcare outcomes for the individual. The method may further comprise at least one of displaying and saving the health care plan for the individual.

In another example embodiment, the method may be performed wherein the inputting of the data related to the individual is through a dedicated computing device.

In another example embodiment, the method may be performed wherein the inputting of the data related to the individual is through a computing device and through a database at least one of owned and operated by a second party.

In another example embodiment, the method may be performed wherein the second party is one of an insurance company, a healthcare provider, an assisted living facility, a memory care facility, a hospice facility, a skilled nursing facility, a pharmaceutical company, a biotechnology company, a remote patient monitoring company, a tele-health company, a government agency, a health and wellness company, a social media company and a healthcare foundation.

In another example embodiment, the method may be performed wherein the quality check reviews for both errors in input data and an amount of data related to satisfy the threshold amount of data.

In another example embodiment, the method may be performed wherein the sorting and analyzing the matching data is performed through a translation table.

In another example embodiment, the method may be performed wherein the healthcare plan is created based upon at least one factor.

In another example embodiment, the method may be performed wherein the at least one factor is cost of healthcare, longevity of the patient, palliative care, age, and a ranked set of healthcare needs.

In another example embodiment, the method may be performed wherein the at least one factor is pre-ranked.

In another example embodiment, the method may be performed wherein the at least one factor is ranked by an algorithm by the artificial intelligence program.

In another example embodiment, the method may further comprise providing access to the plan to at least one of a doctor, a family member, a guardian for the individual, a health care organization, an insurance company, a home healthcare agency and a long-term care facility.

In another example embodiment, the method may be performed wherein the healthcare plan includes expected healthcare costs for the individual.

In another example embodiment, the method may be performed wherein the inputting of the data to the individual is through at least one of memory testing, games, reaction analysis, visual acuity testing, hearing testing, timed exercises, psychological analysis, and personality analysis, hospital admission, hospital readmission and emergency room visits.

In another example embodiment, a method to determine a health care status of at least one individual is disclosed. The method may comprise inputting data related to the at least one individual, the data related to at least one health care statistic for the individual. The method may further comprise performing a quality check of the input data related to the individual. The method may further comprise looping back to the inputting data related to the individual when the quality check indicates that a threshold amount of data of sufficient quality has not been satisfied. The method may further comprise accessing an independent artificial intelligence program. The method may further comprise performing an authentication check of the accessing of the artificial intelligence program to verify that the accessing possesses sufficient authentication for artificial intelligence access. The method may further comprise ending the method when the authentication check to the artificial intelligence fails. The method may further comprise accessing at least one database that contains data related to healthcare patients other than the individual. The method may further comprise creating a list of data to be searched with the at least one database based upon input data from the individual. The method may further comprise searching the at least one database based upon the list of data to be searched to find similar individuals having data withing the at least one database and retaining the matching data. The method may further comprise removing individual personal identifying information from the matching data. The method may further comprise using the artificial intelligence program to sort and analyze the matching data to identify a current healthcare status for the individual.

In another example embodiment, the method may further comprise at least one of saving the status for the individual, printing the status of the individual and displaying the status for the individual.

In another example embodiment, the method may be performed wherein the status for the individual is presented as at least one of a histogram and a numerical score compared to an average individual with similar age.

In another example embodiment, a method to determine a health care status of at least one individual is disclosed. The method may comprise accessing an independent artificial intelligence program. The method may also comprise accessing at least one database that contains data related to healthcare patients other than the individual. The method may also comprise selecting health care data for the at least one individual. The method may also comprise creating a list of individuals with similar health care data to the at least one individual. The method may also comprise using the artificial intelligence program to sort and analyze the matching data to identify a current healthcare status for the individual to achieve a result. The method may also comprise at least one of displaying and printing the result.

In another example embodiment, the method may be performed wherein the artificial intelligence program creates the list of individuals with similar health care data upon at least one factor of age, body mass index, gender, medical affliction, sleep patterns, blood pressure, medications used, medical testing, patient reaction capabilities, mental evaluation and financial capacity.

In another example embodiment, the method may be performed wherein the artificial intelligence program uses at least one database from an actuarial table, a public health records entity, a patient input database, an insurance company database, an assisted living facility, a memory care facility, a home healthcare agency, a hospice facility, a skilled nursing facility, a pharmaceutical company, a biotechnology company, a remote patient monitoring company, a tele-health company, a government agency, a health and wellness company, a social media company, a home health care company, a health non-profit and a healthcare foundation.

In another example embodiment, the method may be performed wherein the at least one individual is a caregiver.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may be varied in many ways. Such variations are not to be regarded as a departure from the disclosure, and all such modifications are intended to be included within the scope of the disclosure.

While embodiments have been described herein, those skilled in the art, having benefit of this disclosure, will appreciate that other embodiments are envisioned that do not depart from the inventive scope. Accordingly, the scope of the present claims or any subsequent claims shall not be unduly limited by the description of the embodiments described herein.

What is claimed is:

1. A method of processing health, wellness and medical data, comprising:
obtaining a first set of health, wellness and medical data related to a first medium;
obtaining a second set of health, wellness and medical data related to a second medium;
inputting the first set of health, wellness and medical data into an artificial intelligence program;
evaluating the first set of health, wellness and medical data with the artificial intelligence program to produce first results;
inputting the second set of health, wellness and medical data into the artificial intelligence program;
evaluating the second set of health, wellness and medical data with the artificial intelligence program to produce second results;
sorting the first results and the second results into database categories;
storing the first results and the second results into a database;
extracting data from the database into a modeling database;
evaluating the modeling database with the artificial intelligence system to produce evaluated results; and
producing at least one report with the evaluated results.

2. The method according to claim 1, wherein the first medium and the second medium are not a same medium.

3. The method according to claim 1, wherein the at least one report includes data related to caregiver performance.

4. The method according to claim 1, wherein the at least one report includes data related to a client health outcome.

5. The method according to claim 1, wherein the at least one report includes data related to creating an agency report.

6. The method according to claim 1, further comprising displaying the at least one report with evaluated results.

7. The method according to claim 1, further comprising saving the at least one report into a non-volatile memory system.

8. The method according to claim 1, wherein at least one of the first medium and the second medium is a handwritten document.

9. The method according to claim 1, wherein at least one of the first medium and the second medium is one of digital information on a wearable device and data for client data.

10. The method according to claim 1, wherein the at least one report indicates an aging in place value for a patient.

11. A method of processing health, wellness and medical data, comprising:
inputting a first set of health, wellness and medical data into an artificial intelligence program;
evaluating the first set of health, wellness and medical data with the artificial intelligence program to produce first results;
sorting the first results into database categories;
extracting data from the sorted first results into a modeling database;
evaluating the modeling database with the artificial intelligence system to produce evaluated results; and
producing an aging in place score for a patient.

12. The method according to claim 11, further comprising displaying the aging in place score for the patient.

13. The method according to claim 11, wherein the evaluating the first set of medical data includes rejecting data that is duplicative of existing medical data in a database.

14. The method according to claim 11, wherein the evaluating includes performing handwriting analysis of a caregiver's notes.

15. The method according to claim 11, wherein the evaluating includes updating a database with evaluated data.

16. The method according to claim 11, further comprising permitting access to proceed with the method based upon a payment system.

17. The method according to claim 11, wherein the evaluating is performed on a cloud-computing arrangement.

18. The method according to claim 11, wherein the inputting of the medical data is at a healthcare facility.

19. An article of manufacture, containing a list of instructions that is readable by a computing apparatus, the list of instructions, at least in part, comprising:
a method of processing health, wellness and medical data, comprising:
obtaining a first set of health, wellness and medical data related to a first medium;
obtaining a second set of health, wellness and medical data related to a second medium;
inputting the first set of health, wellness and medical data into an artificial intelligence program;
evaluating the first set of health, wellness and medical data with the artificial intelligence program to produce first results;
inputting the second set of health, wellness and medical data into the artificial intelligence program;
evaluating the second set of health, wellness and medical data into the artificial intelligence program to produce second results;
sorting the first results and the second results into database categories;
storing the first results and the second results into a database;
extracting data from the database into a modeling database;
evaluating the modeling database with the artificial intelligence system to produce evaluated results; and
producing at least one report with the evaluated results.

20. The article of manufacture according to claim 19, wherein a form of the article of manufacture is a compact disk, a solid-state drive, a computer hard disk, and a universal serial bus device.
